(19) United States
(12) Patent Application Publication
(10) Pub. No.: US 2006/0094001 A1
(43) Pub. Date: May 4, 2006

(54) Method and device for image processing and learning with neuronal cultures

(76) Inventors: Vicent Elisabetta Torre, Trieste (IT); Maria Elisabetta Ruaro, Trieste (IT); Paolo Bonifazi, Trieste (IT)

Correspondence Address: Albert Wai-Kit Chan;Law Offices of Albert Wai-Kit Chan, World Plaza Suite 604, 141-07 20th Avenue, Whitestone, NY, 11357, US

(21) Appl. No.: 10/536,481

(22) PCT Filed: May 23, 2003
(86) PCT No.: PCT/IT03/00317

§ 371 Date: Sep. 16, 2005

(30) Foreign Application Priority Data
Nov. 29, 2002 (IT) RM2002A000604

(51) Int. Cl. C12Q 1/00; C12Q 1/68; G06F 19/00; G06K 9/00

(52) U.S. Cl. 435/4; 435/6; 382/128; 702/19

(57) ABSTRACT

It is disclosed a device for image processing and learning comprising at least a “multi electrode array” (MEA), over which an homogeneous culture of interconnected neurons, so that forming a cell network, is grown on, wherein said MEA is able to stimulate and record the electric activity of said neurons. Methods for image processing and learning utilizing the device are disclosed too.

### TECHNICAL BACKGROUND

Standard silicon devices solve, in a very efficient way, serial problems, but despite their remarkable speed, they are less suitable for solving massive parallel problems, such as those occurring in artificial intelligence, computer vision and robotics¹⁻². Man-made devices are less suitable for massive parallelism because of the difficulty of forming lots of connections between processing units, which biological neurons are ideal for. Despite being slow and often unreliable computing elements³⁻⁵, neurons operate naturally in parallel allowing our brain to solve massive parallel problems. In order to capture basic properties of biological neurons, such as their ability to learn, adapt and their intrinsic parallel processing, Artificial Neural Networks (ANNs) were developed^{1-2,6-8}. ANNs are usually implemented on conventional serial machine losing their original biological inspiration. ANNs can be trained to recognize features and patterns leading to useful and powerful devices, i.e. perceptrons^{6,8}. It is desirable, therefore, to implement ANNs on genuine parallel devices, ideally networks of natural neurons able to learn.

Advances in biocompatibilty of materials and electronics allow to culture neurons directly on metal or silicon substrates, through which it is possible to stimulate and record the electrical neuronal activity⁹⁻¹⁶.

The authors developed a hybrid device for information processing with biological neurons. By using commercially available multi-electrode array (MEA) to interface neuronal cultures, it is possible to process images with two fundamental properties: parallelism and learning. Mapping digital images into the extracellular stimulation of the neuronal culture (in a one by one correspondence between pixels and electrodes) a dynamical low pass filtering of the images is obtained. This processing occurs in just few milliseconds, independently from the dimension of the image processed. Filtered images are obtained by counting and normalizing the supra-threshold evoked events (or extracellular spikes) recorded by the electrodes (pixels). The natural connectivity among cultivated neurons provides the substrate for the massive parallel processing. In addition the neuronal culture can be trained to potentiate the response to simple spatial pattern of stimulation such as an L or a ┐. By applying a strong tetanus with the same spatial profile of the feature to be recognized, learning is induced, due to changes in synaptic efficacy usually referred to as long-term potentiation (LTP) or long-term depression (LTD¹⁷⁻¹⁹.

Filtering and learning can be combined to extract features from processed images. These results open a new perspective for the development of novel hybrid devices composed by biological neurons and artificial elements, i.e. Neurocomputers, providing the ideal machine for massive parallel processing.

### DESCRIPTION OF THE INVENTION

Information processing in the nervous system is based on parallel computation, adaptation and learning. These features inspired the development of Artificial Neural Networks (ANNs), which were implemented on digital serial computers and not parallel processors. Using commercially available multi-electrode arrays (MEA) to record and stimulate the electrical activity from neuronal cultures, the authors have explored the possibility of processing information directly with biological neuronal networks. By mapping digital images, i.e. array of pixels, into the stimulation of the neuronal cultures, it is possible to obtain a dynamical low pass filtering of images within just few milliseconds, and, by subtraction, a band pass filtering of them. Response to specific spatial patterns of stimulation could be potentiated by an appropriate training (tetanization) as consequence of changes in synaptic efficacy. Learning allows pattern recognition and extraction of spatial features in processed images. Therefore neurocomputers, i.e. hybrid devices containing man-made elements and natural neurons, are feasible and may become a new generation of computing devices, to be developed by the synergy of material science and cell biology.

It is therefore an object of the invention a device for image processing and learning comprising at least a “multi electrode array” (MEA), over which an homogeneous culture of interconnected neurons, so that forming a cell network, is grown on, wherein said MEA is able to stimulate and record the electric activity of said neurons.

It is another object of the invention a method for parallel processing a digital image comprising the following steps:
a) mapping a digital image (I_{1,2}(x,y)) (INPUT) having a resolution of 1 or 2 bit (I₁(x,y)) or I₂(x,y)) in the case the image is of 1 or 2 bit respectively) of N×N pixel in voltage pulses of 2 or 4 intensity levels applied to a matrix of N×N integrated electrodes on a multi-electrode array (MEA), where spontaneaously interconnected neurons, so that forming a cell network, are maintained in culture;
b) elaborating the image from said neurons by means of the kernel of convolution:
   h(ρ,σ,t)=A(t)exp((ρ−ρ(t))/2σ(t)²) (1)
   ρ²=x²+y²
c) registering the electric activity of said neurons by means of extracellular MEA electric signals (by voltage) and
d) revealing, for each single electrode and in subsequent time intervals, spikes or firings associated to action potentials generated by said neurons.

Preferably the method comprises a step wherein the firing rate FR(x,y,t) (OUTPUT), measured by the electrode in position (x,y) and during a time interval centered in t, is recorded.

Preferably the INPUT and the OUTPUT are related by the equation:
FR_{1,2}(x,y,t)=I_{1,2}(x,y)**h(ρ,σ,t) (2)
where ** indicates a two-dimensional convolution.

In a particular aspect the INPUT digital image (I₈(x,y)) is defined by 8 bit and is divided into 4 or 8 images (Iₘᵢ), each having 2 or 1 bit respectively, where m is 2 or 1 respectively, according to the equation:

$${I}_{8} ( x , y ) = {{{\sum }_{i}}_{1}}^{8 / m} {I}_{mi} {2}^{m ( i - 1 )} \qquad ( 3 )$$

and each single image Iₘᵢ is filtered indipendently and then reassembled in an unique 8 bit image, wherein the whole process of dividing, filtering and reassembling is according to the equation:

$${{{\sum }_{i}}_{1}}^{8 / m} {2}^{m ( i - 1 )} {I}_{mi} ** h ( ρ , σ , t ) \qquad ( 4 )$$

so that the 8 bit image I₈(x,y) is processed with a 8 bit resolution.

It is a further aspect of the invention a method for digital image processing and learning comprising the following steps:
a) stimulate a matrix of N×N electrodes on a multi-electrode array (MEA), where spontaneaosly interconnected neuronal cells, so that forming a cell network, are maintained in culture, by means of a tetanic stimulation composed by bipolar voltage pulses having a frequency of at least 100 Hz, and having at least a pair of not collinear segments (I_{1,2}(x,y)) (INPUT), in order to induce learning i.e. potentiation;
b) measuring the firing rate FR_{1,2}(x,y,t) evoked by the INPUT image;
c) processing the INPUT image as a 8 bit image according to the equation:

$${{{\sum }_{i}}_{1}}^{8 / m} {2}^{m ( i - 1 )} {FR}_{m , i} ( x , y ) \qquad ( 5 )$$

where FR_{m,i}(x,y) is the measured response to Iₘᵢ(x,y) after the tetanization.

Preferably the INPUT image is larger than 1000×1000 pixel.

In the instant specification terms as “neurons”, “neuronal cells”, “neuronal culture” refer to excitable cells specialized for the transmission of electrical signals, or cellular progenitors thereof.

Stem cell technology can be advantageously used for obtaining a standardized source of neurons. Moreover it could be abdavantageous to automate with appropriate robots all the subsequent procedures necessary for preparing and mantaining neuronal cultures. It is very important to standardize handling of MEAs, neuron deposition on the MEAs and their maintenance. Neurocomputers are likely to be at the basis of a new generation of computing devices, developed by the synergy of material science and cell biology. These computing devices will have human-like capabilities, such as learning, adaptability, robustness and gentle degradation.

### FIGURE LEGENDS

FIG. 1: Mapping an image into the stimulation of a neuronal culture. A: a 6×10 binary digital image of an L used as the stimulation pattern of a neuronal culture grown over a 6×10 MEA manufactured by MCS (B). The neuronal culture obtained from dissociated hippocampal neurons (see Experimental protocol). A magnification of the neuronal culture on the area of the MEA marked by the letters B and C (white rectangle) is shown in the inset C: The electrical activity recorded by the MEA evoked by the electrode stimulation with bipolar voltage pulses of 0.9 V. The silent electrode indicated by the arrow was used as the ground. D: three representative voltage recordings following voltage pulses of 0.3, 0.6 and 0.9 V. E: AFR (see Experimental protocol) recorded by a representative electrode (same as the one used in d) at different voltage stimulation, as indicated in the panel. F: AFR at different repetition rates as indicated in the panel. Data obtained from 50 different trials of the same stimulation. Time 0 corresponds to the voltage stimulation. In e and f a binwidth of 10 msec was used.

FIG. 2: Spread of excitation through the neuronal culture. A shows the electrical activity evoked as function of the distance from the stimulating row of electrodes. The AFR has been measured at each electrode, smoothed over the neighboring electrodes (see Experimental protocol) and averaged by row. From left to right it is shown the AFR calculated in the time windows of 1-6, 4-9, 7-12 and 12-17 msec after the stimulation of the uppermost row of electrodes with a voltage pulse of 0.6 V. Colored points are experimental data from 5 different neuronal cultures and solid lines are theoretical fits with the eq (1). In the first and second panel the fit was obtained by setting ρ equal to 0 and σ was 890 and 1240 respectively. In the third and fourth panel the fits were obtained by setting ρ equal to 920 and 1750, σ equal to 980 and 1130 respectively. B: upper row: images obtained from the processing performed by the neuronal culture in the corresponding time windows; lower row: digital filtering of a binary 6×10 image, with the uppermost row of pixels equal to 1 and 0 elsewhere using the equation (1) with the parameters used for the fitting in different panels in A. AFR in B and C is represented according the color map (see Experimental protocol section) reproduced at the right side of the figure.

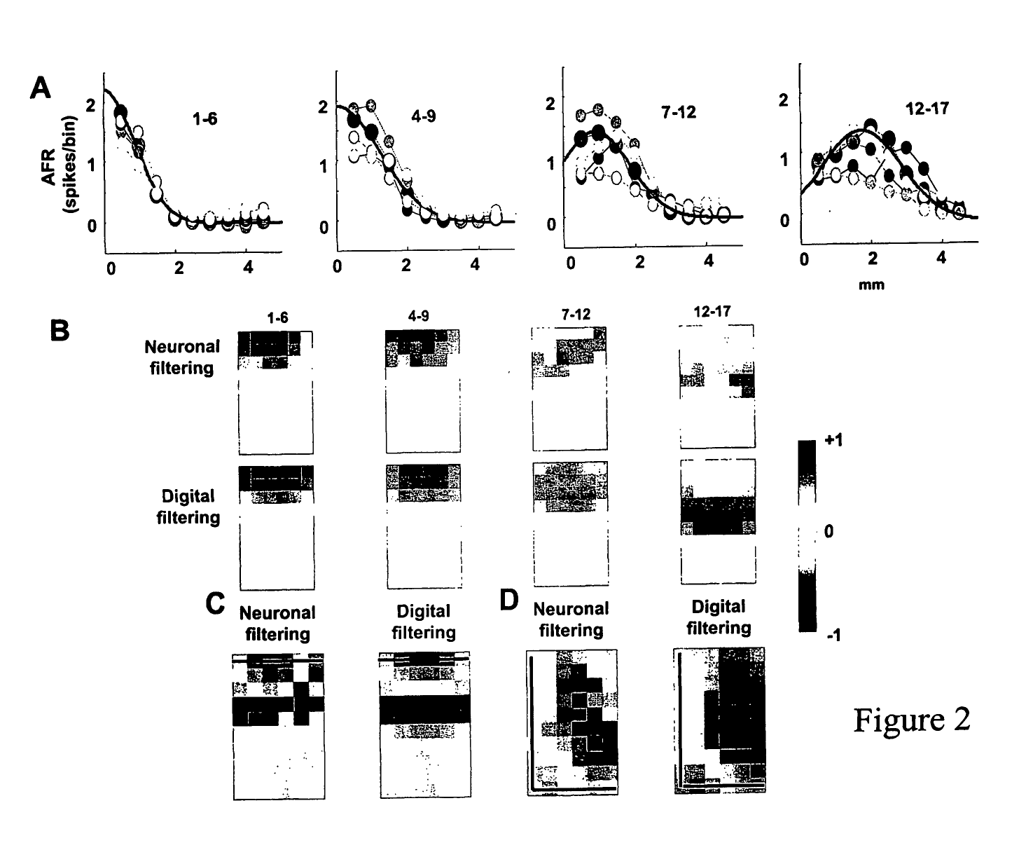

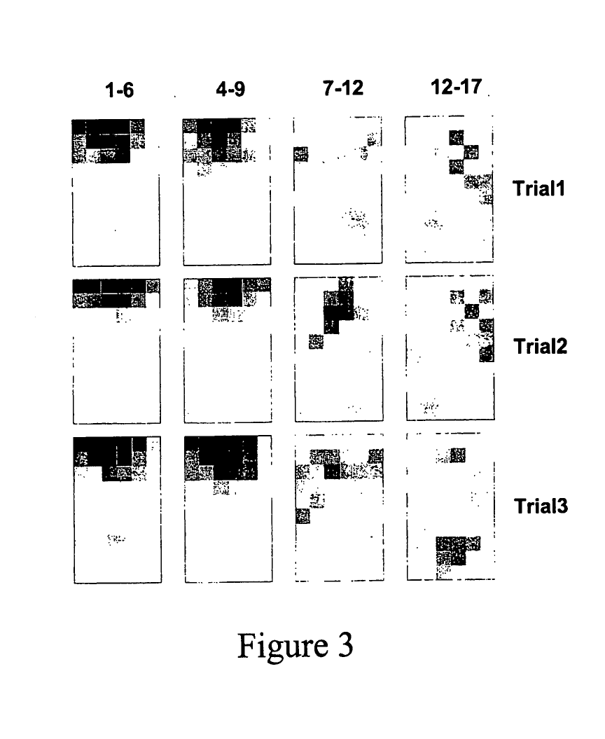

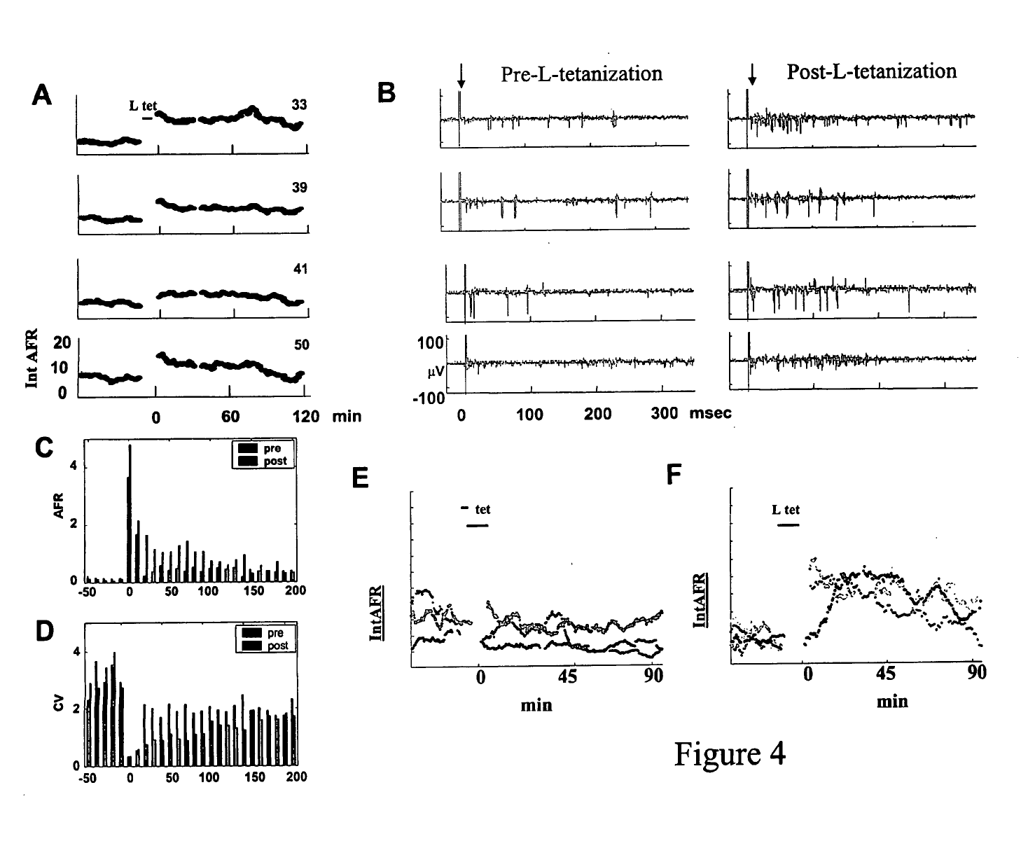

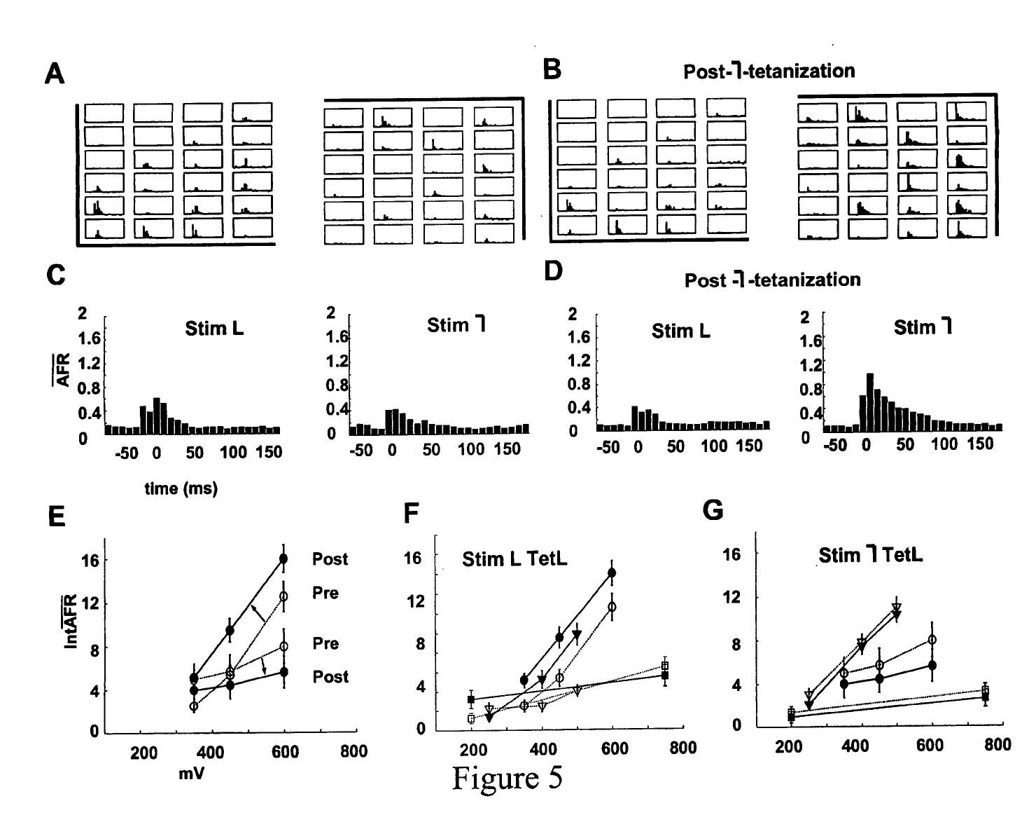

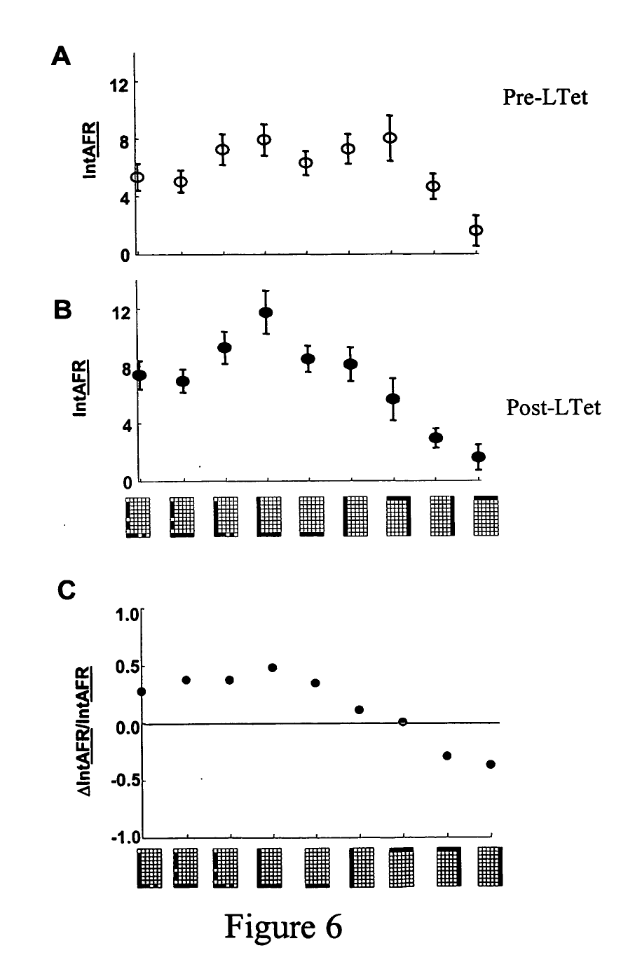

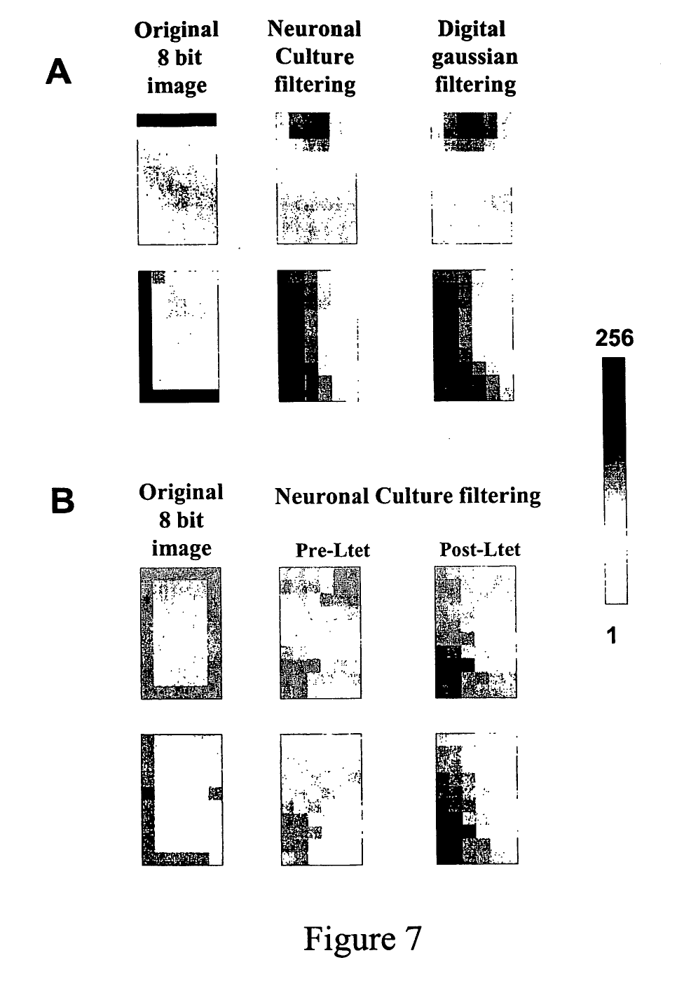

C and D: band pass filtering of the neuronal culture: left panel: band pass filtering of the neuronal culture of a binary image showing an horizontal bar (—), and an L respectively obtained by subtracting the AFRs in the time windows 1-6 and 5-10 msec; right panel: digital filtering obtained by convolving the original binary image with the difference of the two Gaussians fitting the experimental data in the first and second panel of FIG. 2A. The thin bars indicate the stimulated electrodes. Color coding as described in the Experimental protocol section is reproduced at the right side of the figure.

FIG. 3: Reproducibility of image filtering of the neuronal culture: each row reproduces images obtained from a single sweep or trial, in the four time windows, indicated at the top of each column. FR in each image is represented according to the color map (see Experimental protocol section) reproduced at the right side of the figure.

FIG. 4: Induction of LTP in a neuronal culture from ippocampal neurons. A: time dependence of Int AFR prior and after tetanus (indicated by a solid horizontal bar). Int AFR is the integral of AFR from 5 to 100 msec after the stimulation voltage pulse. Each point was obtained from averaging 20 responses to the same stimulation repeated every 4 sec. Tetanus as described in the Experimental protocol. Each panel in B and C refers to the electrode in A with the same number. B: single extracellular voltage response obtained before (left) and after tetanization (right) from the electrodes indicated by the same number in A. Time zero corresponds to the termination of the stimulating bipolar voltage pulse. The large transient at time zero is the residual artifact after its subtraction (see Experimental protocol). C, D: AFR C) and CV (D) before (computed in a time window of 30 minutes before tetanus—in blue) and after (in a time window 30 minutes after tetanus—in red) tetanus recorded at electrode 50. E,F: evolution of IntAFR in different experiments after a tetanus with a spatial profile of a single bar (C) and with a spatial profile of an L (D). In C and D the stimulus had the same spatial profile of the tetanus. In C and D the black points were obtained from the same dish when the tetanus with a bar-shape was first used, followed by L-shape tetanus two hours later.

FIG. 5: Neuronal cultures can learn to distinguish between two different spatial profiles. A: AFRᵢᵢ(t) recorded at 24 electrodes for two stimuli with an L-shaped (left) and ┐-shaped (right) spatial profile. During the experiment a stimulus with an L-shaped and a ┐-shaped profile were alternated to stimuli with the spatial profile of the four bars framing the region of the dish under examination. Stimuli with three different intensities were used (350, 450 and 600 mV), so that the same stimulus was repeated every 36 sec. AFR in A were obtained from 50 individual responses obtained in a time window of 30 minutes before and after tetanus. B: as in A but after a ┐-shaped tetanus. C, D: AFR obtained by averaging in space all the 24 AFR shown in A. The 4 AFR shown refer to the responses to the L and ┐-shaped stimuli before (C) and after (D) tetanus with a ┐ shape. E: dependence of IntAFR from stimulus intensity before (open symbols) and after tetanus (filled symbols) from another experiment with a different dish. IntAFR obtained by integrating AFR from 5 to 100 msec. F: collected data from three different experiments in which the stimulus and tetanus had the same shape. G: collected data from three different experiments in which the stimulus and tetanus had different shape: they were composed by two bars meeting at a different corner. In E, F and G open and closed symbols refer to data collected before and after tetanus respectively.

FIG. 6: Spatial selectivity of LTP. A and B: IntAFR for stimuli with the shape indicated in the abscissa before (open symbols in A) and after tetanus (filled symbols in B) with an L-shaped profile. The voltage intensity of the stimulation was 600 mV. C: relative change of IntAFR produced by the L-shaped profile. Data obtained from those shown in A and B.

FIG. 7: Image processing of 8 bit images. Original 8 bit images are according to eq. (3). A: Low pass filtering of two different 8 bits images. Left panels: The original 8 bits images. Central panels: a low pass filtering of the images, obtained with the neuronal culture. Right panels: a low pass filtering of the images, obtained by a digital convolution of the original 8 bits image, with the Gaussian profile shown in 2A. Color coding as described in the Experimental protocol section is reproduced at the right side of the figure. B: Features extraction: low pass filtering of two different 8 bits images before and after learning. Left column: The original 8 bits images. Central column: a low pass filtering of the images, obtained with the neuronal culture before the tetanization. Right column: a low pass filtering of the images, obtained with the neuronal culture after tetanization with an L-spatial profile. Color coding as described in the Experimental protocol section is reproduced at the right side of the figure. For 1-bit processed images, the values of AFRᵢⱼ(t) were scaled between 0 and 1 by dividing for the corresponding maximal value among all electrodes in the time-window 0-25 ms. 8-bit processed images are then obtained by eq. (4). For features extraction, the values of AFRᵢⱼ(t) obtained after the tetanization were scaled dividing for the same maximal value calculated before the tetanization. 8-bit processed images are then obtained by eq. (5).

### EXPERIMENTAL PROTOCOLS

Neuronal Culture Preparation

Dissection and dissociation: Hippocampus from three-day-old Wistar rats was dissected in ice cold dissection medium (Hanks' modified —Ca2+/Mg2+ free-solution supplemented with 4.2 mM NaHCO₃, 12 mM Hepes, 33 mM D-glucose, 200 μM kinurenic acid, 25 μM APV, 5 μg/ml gentamycin, 0.3% BSA). Slices, cut with a razor blade, were transferred in a 15-ml centrifuge tube and washed twice with the dissection medium. Slices were then treated with 5 mg/ml Trypsin and 0.75 mg/ml DNAseI in digestion medium (137 mM NaCl, 5 mM KCl, 7 mM Na2HPO₄, 25 mM Hepes, 4.2 mM NaHCO₃, 200 μM kinurenic acid, 25 μM APV) for 5 min at RT to perform enzymatic dissociation. Trypsin solution was removed, slices were washed twice with the ice-cold dissection medium, and trypsin was neutralized for 15 min on ice by 1 mg/ml Trypsin inhibitor in the dissection medium. After three washes with the dissection medium, slices were re-suspended in DNAseI (0.5 mg/ml in dissection medium) and mechanically dissociated by several passages through a blue Gilson tip. The cell suspension was then centrifuged at 100 g for 5 min, and pellet was re-suspended in culture medium (MEM supplemented with 0.5% D-glucose, 14 mM Hepes, 0.1 mg/ml apo-transferrin, 30 μg/ml insulin, 0.1 μg/ml d-biotin, 1 mM Vit. B12, 2 μg/ml gentamycin, 5% FCS).

MEA coating: MEA dishes were coated by overnight incubation at 37° C. with 1 ml of 50 μg/ml polyornithine (in water). Dishes were then air-dried and a film of BD-Matrigel (Beckton-Dickinson) was added 20 min before seeding only on the electrode matrix region.

Cell culture: 100 μl of cell suspension was laid on the electrode array of pre-coated MEA at the concentration of 8×10⁵ cells/cm². Cells were let to settle at room temperature for 20 min, then 1 ml of culture medium was added to the MEA and incubated in a 5% CO₂ atmosphere at 37° C.After 48 hours cells were re-fed with neural medium containing 5 μM cytosine-β-D-arabinofuranoside (Ara-C), to block glial cell proliferation, and re-incubated with gentle rocking. Half the medium was changed twice a week. Recordings were performed from 3 weeks after seeding up to 3 months. The same dish could be used for electrical recordings several times and often for almost a month. During electrical recordings, dishes were sealed by a cap manufactured by MCS (MultiChannelSystem). Dishes here used had spacing between each electrode of 500 □m and each metal electrode had a dimension of 30×30 μm.

Maintenance of Neuronal cultures: Neuronal cultures were kept in an incubator providing a controlled level of CO₂, temperature and moister. When a dish was moved from the incubator to the electrical recording system, the neuronal culture was allowed to settle for about 2 hours so to reach a stationary state. In this period, often a run down of the spontaneous and evoked electrical activity was observed over 2-4 hours, which can account for the decrease of the response of the neuronal culture to stimuli different from that used for the tetanus (see FIG. 5-6). Several hours before electrical recordings, dishes were sealed by a cap manufactured by ALA Science and distributed by MCS (MultiChannelSystem) so decreasing the observed run down. After termination of the experiment, usually between 3 and 10 hours, the dish was moved back to the incubator. The same dish could be used for an other experiment in the following days and often over a month. In some cases the same dish was used for more than four different experiments.

Electrical Recordings and Electrode Stimulation

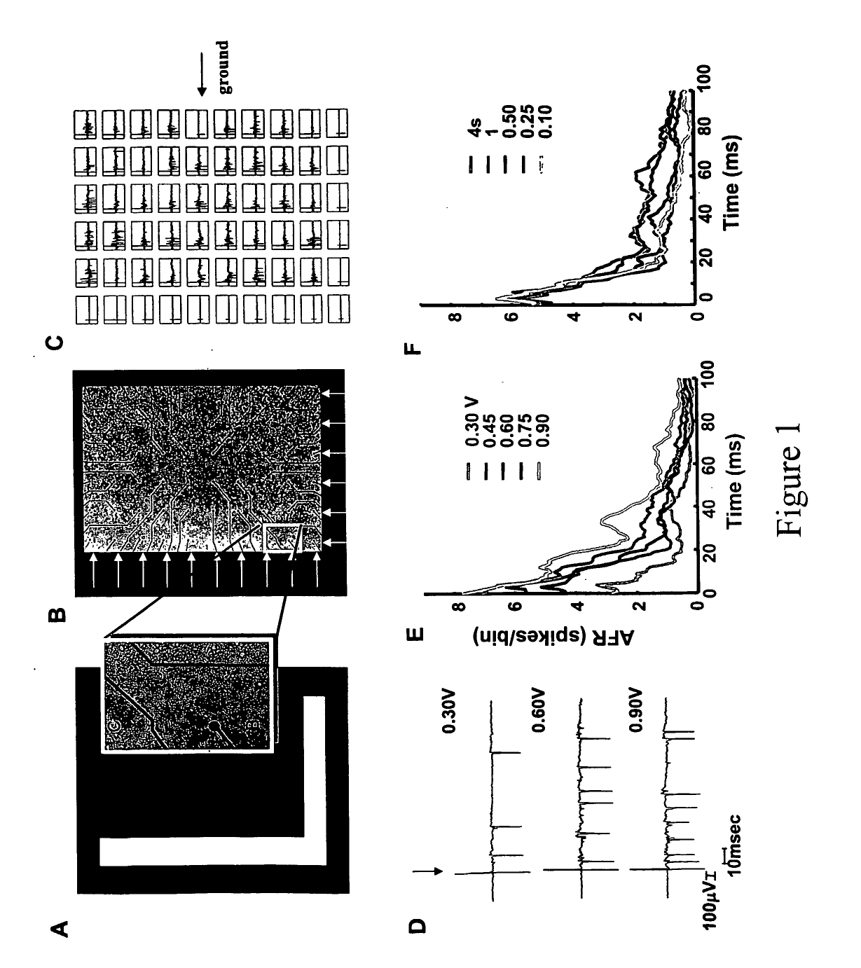

The system commercially supplied by MultiChannel Systems was used for electrical recording. In the present report we refer to a 6×10 microelectrode array, with a 500 μm spacing between adjacent electrodes. Each titanium-nitride microelectrode has a 30 μm diameter circular shape; its frequency-dependent impedance is of the order of 100 kΩ at 1 kHz. Through gold contacts it is connected to a 60 channel, 10 Hz-3 kHz bandwidth pre-amplifier/filter-amplifier (MEA 1060-AMP) which redirects the signals toward a further electronic processing (i.e. amplification and AD conversion), operated by a board lodged within a high performance PC. Signal acquisitions are managed under software control. A thermostat (HC-X) maintains the temperature at 37° C. underneath the MEA. The MEA provided by MCS is able to digitize in real time at 20 kHz all voltage recordings Vᵢⱼ obtained from the 60 metal electrodes. One electrode was used as ground (see FIG. 1C). Sample data were transferred in real time to the hard disk for later processing. Each metal electrode could be used for recording or for stimulation, but the present MCS system does not allow a computer-controlled switch from one mode to the other. Therefore, during a trial, each electrode can be used either for stimulation or recording. Voltage stimulation Sᵢⱼ consisted in bipolar pulses lasting 100 microseconds at each polarity, of amplitude varying from 200 mV to 1 V, injected through the STG1004 Stimulus Generator. An artifact lasting 5-20 msec caused by the electrical stimulation was induced on the recording electrodes. This artifact was removed from the electrical recordings during data analysis. When 6 stimuli with a different spatial profile were used each of them delivered at three different voltages, the same stimulus was repeated every 36 seconds.

Tetanus: The tetanus consisted in 40 trains of bipolar pulses of ±900 mV lasting for 100 μsec delivered every 2 seconds. Every train consisted in 100 pulses at 250 Hz. Test stimuli before and after tetanus were delivered every 2 seconds.

Data analysis: Acquired data were analyzed using the software MatLab (The Mathworks, inc.).

Artifact removal: The artifact at each electrode and for each pattern of stimulation was estimated and subtracted from the voltage recordings. The artifact was estimated in the following way: for each pattern of stimulation and at each electrode the voltage response averaged over all trials (typically 50) was computed and was fitted by 2 polynomials of 9th degree. The 2 polynomials fitted the data in the time window of 0.5-25 ms and 7.5-100 ms after the stimulation respectively. The first polynomial was used to evaluate the artifact in the time window from 0.5 to 7.5 msec, while the second in the time window from 7.5 and 82.5 msec. The artifact, so evaluated, was subtracted from the original voltage signal.

Computation of firing rate (FR) : Let Vᵢⱼ be the voltage recorded at electrode (ij) and σᵢⱼ be the standard deviation of the noise computed considering a period of at least 1 sec where no spikes were visually observed. The firing rate FRᵢⱼ(t) at time t=(t₁+t₂)/2 is the number of all level crossings of Vᵢⱼ above a threshold set as 5*σᵢⱼ computed in a time window between t₁ and t₂. This FRᵢⱼ(t) counts spikes from different neurons, making a good electrical contact with electrode (ij). The σᵢⱼ of the noise ranged for individual electrodes from 3 to 6 μV. The average firing rate AFRᵢⱼ(t) was computed by averaging FRᵢⱼ(t) over all trials. Otherwise stated AFR(t) was computed on binwidth of 10 msec. The coefficient of variation CV was similarly computed as the standard deviation of FRᵢⱼ (t).The average firing rate AFR(t) averaged over a set of electrodes AFR(t) was obtained by averaging AFR(t) over a set of different electrodes, so to have a simple measure of the overall evoked firing rate. Also the integral of AFR (t) over a time window between 5 and 100 sec was computed (IntAFR). This quantity was used to compare the effect of tetanus on the global response evoked by stimuli with a different intensity (see FIG. 5E,F and G).

Image Processing

MEAs with at least more than 54 electrodes providing electrical recordings of clear spikes were used for image processing. Given an image Iᵢⱼ of M×N pixels and a MEA with M×N electrodes, the gray level of pixel (ij) of 1 is converted into an appropriate voltage stimulation Sᵢⱼ of electrode (ij). The MEA provides the voltage signals Vᵢⱼ composed by action potentials or spikes produced by the neurons. The processing of the image Iᵢⱼ is the set of outputs FRᵢⱼ (t), so that, at different times t there is a different processing of the original image Iᵢⱼ.

Mapping Iᵢⱼ into Sᵢⱼ. Let V_{1/2} be the voltage stimulation evoking half of the maximal AFR 10 msec after the onset of the voltage pulse. If Iᵢⱼ is a binary image, i.e. if its gray levels are either 0 or 1, then Sᵢⱼ will be 3/2*Vᵢⱼ if Iᵢⱼ is 1, 0 otherwise. If Iᵢⱼ is a 2 bits image, i.e. if its grey levels are either 0, 1, 2 or 3, then Sᵢⱼ will be 0 if Iᵢⱼ is 0, Sᵢⱼ will be ½*V_{1/2} if Iᵢⱼ is 1, Sᵢⱼ will be V_{1/2} if Iᵢⱼ is 2 and Sij will be 3/2*V_{1/2} if Iᵢⱼ is 3.

Filling silent electrodes and smoothing. When one electrode (i, j) is silent, i.e. no spikes can be recorded, the corresponding hole in the processed image is filled in by assigning to FRij (t) the value obtained averaging the firing rate from neighboring electrodes—i.e. electrodes at a distance of 500 μm. FRᵢⱼ (t) of stimulated electrodes was determined by extrapolation from the neighboring active electrodes using eq (1). All processed images had at most 3 silent electrodes, including the one used as ground. Often, the value of FRᵢⱼ (t) was smoothed over the neighboring electrodes (i−1, j) (i+1,j), (i, j−1) and (i, j+1). The FRᵢⱼ (t) of electrodes used for stimulation was extrapolated from the value of neighboring electrodes.

Processing of 8 bit images. The 8 bits image 1₈(x, y) was decomposed as

$${{{\sum }_{i}}_{1}}^{8} {I}_{i1} ( x , y ) {2}^{( i - 1 )}$$

where Iᵢ₁(x,y) is a 1 bit image, or

$${{{\sum }_{i}}_{1}}^{4} {I}_{i2} ( x , y ) {2}^{2 ( i - 1 )}$$

where x,y) is a 2 bits image. The 8 Iᵢ₁I(x,y) 1-bit images or the 4 Iᵢ₂(x,y) 2-bit images are processed as described above and their output was summed as described in eq (4).

Output color coding. Processed images FRᵢⱼ(t), AFRᵢⱼ(t) or their combination (for band pass filtering and/or for 8-bits processing) were displayed using a standard color coding procedure.

For low pass filtered images, the values of FRᵢⱼ(t) (FIG. 3) or AFRᵢⱼ(t) (FIG. 2B, upper row) were scaled between 0 and 1 by dividing for the corresponding maximal value among all electrodes in the time-window 0-25 ms. For features extraction, (FIG. 7B) the values of AFRᵢⱼ(t) obtained after the tetanization were scaled dividing for the same maximal value calculated before the tetanization. Digitally low pass filtered images (FIG. 2B lower row) were first scaled between 0 and 1 dividing for their maximal value, and then multiplied for the maximal value of the corresponding re-scaled image FRᵢⱼ(t).

For band pass filtered images (FIG. 2C, D left panels)—obtained as the difference of FRᵢⱼ (t) or AFRᵢⱼ (t) at two different times—the resulting output was scaled between −1 and +1, dividing for its maximum absolute value.

For digitally band pass filtered images (FIG. 2C, D right panels)—obtained as the difference of digitally low pass filtered images re-scaled as above—the resulting output was scaled between −1 and +1, dividing for its maximum absolute value.

The color map (of 256 colors) was always scaled between −1 and 1.

For 8-bits processed images (FIG. 7), the values of AFRᵢⱼ(t) scaled as above were used and the color map (of 256 colors) was scaled between −256 e 256. Therefore, with this coding, the processing of images at 1,2 or 8-bits has the same map, i.e. the output has 256 different colors. If desired, a different coding can be used.

Results

MEAs with 60 or more electrodes can be obtained from research centers or bought from companies 9-16 MEAs are fabricated with different geometry of electrodes, such as a regular square grid, with a spacing between electrodes varying between 50 to 500 μm. Individual electrodes are usually covered by a thin layer of platinum and have sides ranging from 10 to 30 μm. The great majority of presently available MEAs and arrays of CCD camera share the same geometry of a square grid. This observation inspired the design of a device for processing images where the computing element is a neuronal culture grown on a MEA: the image is mapped to the voltage stimulation of a neuronal culture and the evoked electrical activity is taken as the output of the new device.

The Device

The proposed device is now described in details. An image Iᵢⱼ of M×N pixels, (FIG. 1A) with the usual square geometry is coded into the input of a MEA with M×N electrodes, (FIG. 1B), so that the gray level of pixel (i, j) of Iᵢⱼ is converted into an appropriate voltage stimulation Sᵢⱼ of electrode (i, j) (see Experimental protocol). The output of the device is composed by the voltage signals Vᵢⱼ recorded with the MEA (FIG. 1C). These signals are composed by action potentials or spikes produced by the neurons in the culture (shown in the inset of panel B), and their statistics is used to obtain a coding of the processed image. More specifically, the output of the device is FRᵢⱼ (t), i.e. the firing rate of all neuroris recorded at electrode (i, j) in the time window between t−Δt and t+Δt following the electrode stimulation at time 0. In this way, for each stimulation Sᵢⱼ coding for image Iᵢⱼ, a set of outputs FRᵢⱼ (t) representing the processing of Iij at time t is obtained. In what follows, it will be shown that a neuronal culture of rat hippocampal neurons (see Experimental protocol) can be used to process digital images.

Dynamic range, cycle time and reproducibility of the response of the proposed device were investigated by stimulating a row of electrodes of the MEA with the same voltage pulse. This stimulation corresponds to a black image with a bright narrow bar. Brief bipolar voltages lasting 200 μsec were used and their amplitude was progressively increased from 300 mV to 900 mV. Spikes recorded at one site increased in frequency and often spikes with a different shape, produced by a different neuron, appeared (FIG. 1D). The average firing rate (AFR) of all detected spikes (see Experimental protocol) increased with the voltage stimulation (FIG. 1E). The dynamic range of the AFR, however, was rather narrow: usually no spikes were evoked by voltage pulses below 200 mV and, a saturating maximal response, was evoked with voltage stimulation of about 1 V. In the great majority of the experiments, it was possible to distinguish reliably 4 levels of evoked activity, indicating that the neuronal culture could code 2 bits.

In order to determine the cycle time of the new device, the same stimulation was repeated at intervals from 100 msec to 10 seconds. With a repetition rate higher than 1 or 2 seconds the AFR had two components: one which was evoked with a delay of very few msec lasting for about 15 msec, followed by a second component lasting for 100 msec or so. The amplitude of the first component was not significantly affected by decreasing the repetition time from 10 seconds to 0.1 msec (see FIG. 1F). The amplitude of the second component was clearly depressed at short repetition times and it was stable for repetition times higher than 4 seconds. Therefore, the new device can process 2 bits with a cycle time varying between 0.25 and 10 Hz, depending whether the first or second component in the response is considered.

Filtering Properties of the Neuronal Culture

The neuronal culture grown on the MEA constitutes a two-dimensional network and its filtering properties are better analyzed by using a long bar as a spatial stimulus. In this way given an homogenous culture, the characterization of a two-dimensional network is reduced to the understanding of a much simpler one-dimensional problem: the six electrodes of the upper row were used for stimulation and the AFR evoked in each electrode was measured, smoothed over the neighboring electrodes (see Experimental protocol) and averaged by row. At early times, i.e. in the time window between 1 and 6 msec (FIG. 2A) the computed AFR decayed spatially across the neuronal culture as a Gaussian function with a standard deviation σ of 890 μm corresponding to 1.8 pixels (solid line in first panel of FIG. 2A). In the time window between 4 and 9 msec (FIG. 2B) the electrical activity decayed with a similar Gaussian function but with a larger σ of 1240 μm corresponding to 2.5 pixels (solid line in the second panel of FIG. 2A). A very similar decay and spread of electrical excitation was observed consistently in the great majority of neuronal cultures, as shown by collected data from 5 different dishes in FIG. 2A.

After 10 msecs or so the peak of the evoked AFR moved away from the stimulated electrodes and was described by a Gaussian function centered at a distance ρ from the stimulated electrodes (third panel in FIG. 2A). After 15 msec the evoked electrical activity decayed even further, maintaining a Gaussian-like profile (last panel in FIG. 2A). The same qualitative behavior was observed in all neuronal cultures (see different symbols in FIG. 2A), but the speed by which the electrical activity moved from the stimulating electrodes varied between 70 to 250 μm/msec.

The processing of the neuronal culture of the image Iᵢⱼ composed by a bright bar of 6 pixels in the upper part is represented by the color coding of the AFR, shown in the upper part of FIG. 2B. In the lower part of FIG. 2B are shown the digital convolutions of Iᵢⱼ with a Gaussian kernel with a standard deviation or corresponding to 1.8 and 2.5 pixels (first and second panels) respectively. The similarity of images shows that the neuronal culture indeed performs a Gaussian low pass filtering. At the later times of 9.5 and 14.5 msec, FRᵢⱼ (t) (third and fourth panels) is a displaced low pass filtering of the original image. The possible computational relevance of this displacement will be discussed in another publication.

Experiments where a row (or a column) of electrodes or individual electrodes were stimulated indicate that the spatial-temporal processing of the neuronal culture is in first approximation spatially invariant and can described by a radial impulse response.
h(ρ,σ,t)=A(t)exp((ρ−ρ(t))/2σ(t)²) (1)
ρ²=x²+y²

i.e. a usual Gaussian function or kernel, centered on □(t) and with a time varying variance σ²(t). Therefore, given a 1 or 2 bits image I_{1,2} (x, y) the output of the proposed device FR_{1,2} (x,y, t) varies with time and is:
FR_{1,2}(x, y,t)=I_{1,2}(x,y)**h(ρ,σ,t) (2)

Where ρ and σ depend on time and ** indicates a two-dimensional convolution. Indeed, the third image in the lower part of FIG. 2B obtained by convolving Iᵢⱼ with the kernel (1) having the values of 1250 μm and 850 μm for ρ and σ respectively shows the same features of that in the upper part representing the experimentally observed neuronal filtering.

Reliability of the Device

Unlike silicon devices, biological neurons are affected by a significant noise and it is essential to evaluate the reliability of the proposed device. For identical stimulations the number of evoked spikes was slightly variable, but the first evoked spikes had a very small jitter of about 1 msec. At the peak of the evoked response, the coefficient of variation (CV) of the total number of spikes—computed with a binwidth of 10 msec—was always small around 0.2 (see FIG. 4D). At later times the CV increased to about 1 and even more. At the larger binwidth of 50 msec the CV was always less than 0.5. The CV of the evoked response did not change significantly with the distance of the recording electrode from the stimulation site. FIG. 3 illustrates images obtained from three single trials when the uppermost row of electrodes was stimulated with the same voltage pulse of 600 mV. At early times (see the first and second column) processed images are rather similar. Later than 10 msec processed images differ from trial to trial, consistently with the high CV of the electrical recordings (FIG. 4D) and the larger variability among different neuronal cultures (see last panels in FIG. 2A).

Given the spatio-temporal filtering of eq (1), during the first msec, following the electrical stimulation, when ρ(t) is close to zero and σ²(t) increases, it is possible to perform very quickly a low and band pass filtering of an image. In the time window σ of about 890 μm, but 2 or 3 msec later with a larger value of a of 1240 μm. The two filters are low pass, but their difference is bandpass. Bandpass filtering of binary images representing simple characters such as an L and—obtained with the neuronal culture are shown in FIGS. 2C and D. The comparison with the same filtering performed by a digital computer shows that the device operates as intended.

Neuronal cultures obtained from different mice and cultivated in different dishes had a variable number of electrodes providing good electrical recordings ranging from 30 to 54. All neuronal cultures with a sufficient number of electrically active electrodes so to allow a quantitative characterization of the filtering of the neuronal network, i.e. larger than 40, had the same behavior illustrated in FIG. 2A. At early times the spread of electrical excitation was approximately a gaussian function with a standard deviation increasing from about 800 to 1200 μm in 3 or so msec. At later times the behavior of different neuronal cultures (FIG. 2A) was more variable as the response of an individual culture (see FIG. 2C).

These data show that immediately after the voltage stimulation there is a “good” time window during which the processing of the neuronal culture is reproducible leading to a reliable computation. This reproducibility is observed among trials from the same neuronal culture (see FIG. 3) and in different cultures (see FIG. 2A). Outside this “good” time window there is a significant variability in different trials and cultures.

Learning

Having seen that neuronal cultures can be used to filter images, the next question to answer is: is it possible to induce learning in the neuronal culture? If so is it possible to train the neuronal culture to recognize a specific spatial pattern?

In order to answer to this question a neuronal culture, was stimulated every 2 seconds with bipolar voltage pulses (see Experimental protocol and figure legend) having an L-shaped spatial profile. The voltage stimulation was applied repeatedly for at least one hour, so to have a good statistics of the evoked electrical activity, monitored by computing the average firing rate (AFR) over 20 identical trials and by integrating this quantity over a time window between 5 and 100 msec after the stimulus (IntAFR). Following an L-shaped tetanus (see Experimental protocol), the IntAFR evoked by a stimulus with the same Lshape was significantly increased for 1 hour as shown at four representative electrodes (FIG. 4A). The increase of the evoked electrical activity was also evident by visual inspection of individual traces (FIG. 4B). The AFR at one representative electrode after the tetanus was prolonged and the corresponding CV remained small, i.e. less than 0.5 for almost 20 msec (FIG. 4C,D).

This long lasting increase in evoked electrical activity or LTP was not observed when the frequency of tetanization was lower than 100 Hz. LTP could be induced again in the same neuronal culture after sitting for a few days in the incubator.

When tetanus with a spatial profile of a simple bar was used, a clear LTP was never observed and in some occasions a slight run down of the overall response was observed (see red symbols FIG. 4E). On the contrary, when the tetanus had the spatial profile of two perpendicular bars the overall response was potentiated (see FIG. 4F). In one dish tetanus with a a spatial profile of a simple bar was first used and after 2 hours an L-shaped tetanus was applied inducing a clear LTP (black symbols in FIGS. 4E and F). We also observed that LTP could be induced again in the same neuronal culture after sitting for days in the incubator (see cyan and yellow simbols in FIG. 4F).

The difference in the LTP induction between the simple bar and the L-shaped could be explained if we consider the requirement of pairing i.e. the simultaneous depolarization of pre and post-synaptic neurons, for LTP induction^{17,18}. Indeed, with a stimulation composed by two perpendicular bars, each bar evokes a clear electrical activity over all recording electrodes due to the propagation described in FIG. 2. Therefore the stimulation of the two bars will depolarize simultaneously many pre and post synaptic neurons, providing the basis for the pairing required for LTP induction.

As LTP can be induced in a neuronal culture, it is necessary to establish its spatial structure and specificity and therefore the electrical activity evoked by stimuli with different spatial profiles was compared. Initially an L-shaped and an ┐-shaped stimuli evoked a diffuse response with a comparable number of action potentials (see left and right panel in FIG. 5A). Indeed, the firing rate averaged over different trials and over the recording electrodes (AFR) were very similar (compare left and right pannels in FIG. 5C). After an ┐-shaped tetanus, the response evoked by the L-shaped stimulus slightly decreased. On the contrary, the response to the ┐-shaped stimulus significantly increased (compare right pannels in FIG. 5B and in FIG. 5D). After the tetanus the AFR for the two stimuli was significantly different: the response to the ┐-shaped stimulus was more than twice larger than that evoked by the L-shaped stimulus (compare left and right pannels in FIG. 5D).

The overall response of the neuronal culture to a given stimulus prior and and after tetanization was quantified by the integral of AFR in a time window from 5 to 100 msec (IntAFR, FIG. 5E).For the three stimulus intensities of 350, 450 and 600 mV evoking a significantly different value of IntAFR, the ┐-shaped tetanus clearly potentiated the response evoked by the ┐-shaped stimuli. IntAFR evoked by the L-shaped stimuli slightly decreased, probably for a spontaneous rundown of the evoked response often observed when the neuronal culture was moved from the incubator to the recording system. When the tetanizing stimulus had the spatial profile composed by two perpendicular bars meeting in a corner, IntAFR for a stimulus with the same spatial profile always increased (FIG. 5F), but not for a stimulus composed by two similar bars, but meeting in a different corner (FIG. 5G). LTP could be evoked in the same neuronal culture in several experiments performed in different days over a period of 6 weeks and was observed in at least half a dozen of different neuronal cultures after 25-70 days of cultivation.

If the neuronal culture can be trained to recognize an L-shaped stimulus from a ┐-shaped stimulus it is important to analyse its selectivity and verify whether its response degrades gently with the corruption of the stimulus. The value of IntAFR for stimuli with a different spatial profile before (open symbols) and after an L-shaped tetanus (filled symbols) are compared in FIGS. 6A and B.

Prior tetanus the response of the neuronal culture was not specific to the spatial profile of the stimulus (FIG. 6A). On the contrary, after tetanus with an L-shaped profile, the neuronal culture preferentially responded to stimuli ressembling an L: indeed after tetanus IntAFR was significantly larger for stimuli with a spatial profile similar to an L-shape (FIG. 6B). The relative change of IntAFR after tetanus, was clearly selective (FIG. 6C) and showed a positive values for similar spatial profiles.

The previous results indicate that LTP can be induced by a tetanus with a spatial profile of anL and that the neuronal culture has learned to recognize the L.

Image Processing of 8 bit Images and Feature Extraction

The neuronal culture can be used also for processing digital images at 8 bits. Let I₈(x,y) be an image with 8 bits gray levels at location (x,y). Then I₈(x,y) can be decomposed as:

$${I}_{8} ( x , y ) = {{{\sum }_{i}}_{1}}^{8 / m} {I}_{mi} ( x , y ) {2}^{m ( i - 1 )} \qquad ( 3 )$$

where m is equal to 1 or 2 according to the number of bits coded by the single processed image Iₘᵢ. Given this decomposition, the processing of an 8 bit image is obtained as:

$${{{\sum }_{i}}_{1}}^{8 / m} {2}^{m ( i - 1 )} {I}_{mi} ( x , y ) ** h ( ρ , σ , . t ) \qquad ( 4 )$$

By processing with the neuronal culture independently the 4 2-bits images or the 8 1-bit images a low or a band pass filtering of an 8 bits image is obtained. A low pass filtering of the original 8 bit images (FIG. 7A left panels), obtained by the neuronal culture and by a digital filtering with a Gaussian function, are shown in the central and right panels respectively of FIG. 7A. The high similarity of images in the central and right panel show that the proposed hybrid device can process also 8 bits images. After a neuronal culture has learned, its temporal-spatial filtering is different. First of all it is not anymore spatially invariant and therefore cannot be described by a temporal and spatial convolution as in eq (4). Indeed the firing rate FR_{1,2}(x,y,t) evoked by a given image I_{1,2} (x,y) cannot be predicted from eqs. (1), (2) but must be measured. The processing of an 8 bit image is obtained as:

$${{{\sum }_{i}}_{1}}^{8 / m} {2}^{m ( i - 1 )} {FR}_{m , i} ( x , y ) \qquad ( 5 )$$

where FR_{m,i} (x,y) is the measured response to Iₘᵢ(x,y) after the tetanization. Having lost spatial invariance the device is now able to extract a specific pattern from a complex image. When the neuronal culture has learned to recognize an L (see FIG. 7B), the processing of original images (FIG. 7B left column) is modified (see central column of FIG. 7B) and becomes tuned and selective to L-shaped stimuli (right column of FIG. 7b). It is evident that after learning the neuronal culture is able to extract the L from the rest of the image, in both processed images The upper image shows clearly that the neuronal filtering is symmetric before learning and becomes asymmetric after allowing the extraction of the learned/potentiated feature.
Discussion

It has been demonstrated that by growing neuronal cultures over multi electrode arrays (MEA), a new computing device is obtained, composed by biological neurons and metal electrodes.

The biophysical mechanisms underlying the low-pass and band-pass filtering of digital images, here described, originate from membrane properties of cultivated neurons and their mode of interaction. The generation of action potentials is controlled by “threshold” effects due to constraints on multiple voltage-dependent channels and inactivation of voltage-dependent Na-dependent channels. Synaptic properties limit and shape the propagation of action potentials in the culture. The combination of these biophysical mechanisms determine the exact parameters of the filtering

There are four major advantages in this new device, possibly the first prototype of a neurocomputer. Firstly the spontaneous formation of multiple connections between neurons provides the most obvious substrate for massive parallel processing necessary for the next generation of computing devices. Second, as a consequence of this massive parallel processing, low-pass and band pass filtering can be obtained in less than 10 msec, irrespective of the size of the image to be processed and in sharp contrast with serial computing devices. In fact the proposed device is potentially able to process large digital images (2000×2000 pixels) faster than most of today's digital computers. Thirdly, it is possible to induce LTP in neuronal cultures (FIG. 4-7) which can be trained to recognize spatial patterns. This learning is best obtained by using a strong tetanus with a frequency larger than 200 Hz, in which tetanizing bursts are applied every 2 seconds or so. Learning requires also a significant pairing, i.e. the simultaneous activation of presynaptic and postsynaptic neurons. This is better obtained by using complex patterns of tetanization, possibly encompassing two orthogonal bars (FIG. 4-5). Fourthly, the use of biological neurons as computing devices opens a new avenue in which computer science can capitalize from the expertise and technology of cell biology and genetic engineering. Stem cell technology²⁰⁻²² could provide a standard source of neurons eliminating the variability intrinsic to individual mouse, possibly leading to computing devices with a much higher reliability. Stem cell technology could provide also populations of neurons with specific properties, releasing selected neurotransmitters so to construct neuronal cultures with controlled ratios of inhibitory and excitatory neurons. The possibility of guiding neuronal growth along specific spatial directions²³⁻²⁶ will allow the fabrication of large variety of spatial filters, imitating the receptive field properties of neurons in early visual areas²⁷.

The utility and advantage of the proposed device and possibly of all neurocomputers, depends on the size of parallel processing. The proposed device will give no advantage in processing small images, which can be more accurately processed by standard digital computers. It becomes useful possibly providing better performances than digital computers, when very large images have to be processed larger than 1000×1000 pixels. Such image processing, however, requires the development of MEA with more than 1 million of electrodes. Besides the development of MEA with a very high number of electrodes and the solution of all the interface problems, an efficient use of neurocomputers requires also an appropriate computational framework. Biological neurons are slow and not highly reliable computing elements, but they naturally work in parallel. They are ideal for the solution of massively parallel problems, where the reliability of a single computing element is not critical. Biological neurons and probably all neurocomputers are not suitable to imitate a Turing machine²⁸ i.e. a serial and precise computing device. An efficient use of neurocomputers requires a new computational framework not based on the Touring machine, as usual digital computers do.

The training procedure, by which a Neurocomputer learns to recognize a spatial feature, is simply an appropriate tetanus. As a consequence programmability of this kind of Neurocomputer is almost trivial, in sharp constrast with networks of silicon devices where the complexity of programming is remarkable and possibly a major limitation for their use⁸. After the decline of LTP the Neurocomputer can be trained to learn a new pattern and therefore can be reprogrammed and reusable. One of the major attractions of neurocomputers, is the possibility of using all the adaptability of biological neurons, originating from billions of years of evolution. The exploitation of LTP, as here demonstrated, and of LTD, may provide a natural implementation of algorithms based on artificial neural networks (ANN)⁶⁻⁸.

### REFERENCES

1. Marr. Vision: A Computational Investigation into the Human Representation and Processing of Visual Information. San Francisco. Calif.:Freeman & Co., (1982)
2. Rumelhart, D. E. and J. L. McClelland. Explorations in Parallel Distributed Processing. MIT Press. Cambridge, Mass. (1988)
3. Nicholls J. G., Wallace, B. G., Martin, A. R., and Fuchs P. A. From Neuron to Brain: A Cellular and Molecular Approach to the Function of the Nervous System 4^{TH} edition. Sinauer Associates, Incorporated (2000)
4. Shadlen M N, Newsome W T Noise, neural codes and cortical organization. Curr OpinNeurobiol 4: 569-579 (1994)
5. Zoccolan D, G. Pinato & Torre V. Highly variable spike trains underlie reproducible sensory-motor responses in the medicinal leech. J Neurosci. (in the press) (2002).
6. Introduction to the Theory of Neural Computation, Hertz J., Krogh A., Palmer R. G., (Santa Fe Institute Studies in the Sciences of Complexity. Lecture Notes, Vol 1) (1991).
7. Hopfield, J. J. Neural networks and physical systems with emergent collective computational abilities. Proc. Natl. Acad. Sci. USA 79, 2554-2558 (1982).
8. Marvin L. Minsky, Seymour Papert. Perceptrons: An Introduction to Computational Geometry, MIT Press. (1988).
9. Gross, G. W., Rieske, E., Kreutzberg, G. W. & Meyer, A. A new fixed-array multimicroelectrode system designed for longterm monitoring of extracellular single unit neuronal activity in vitro. Neurosci. Lett. 6, 101-105 (1977)
10. Pine, J. Recording action potentials from cultured neurons with extracellular microcircuit electrodes. J. Neurosci. Methods, 2, 19-31 (1980)
11. Novak, J. L. & Wheeler, B. C. Recording from tbe aplysia abdominal ganglion with a planar microelectrode array. IEEE Trans, Biomed. Eng. 33 196-202 (1986)
12. Jimbo Y. & Kawana A. Electrical stimulation and recording from cultured neurons using a planar electrode array, Bioelectrochem. Bioenergetics 29, 193-204 (1992)
13. Martinoia, S., Bove, M., Carlini, G., Ciccarelli, C., Grattarola, M., Storment, C., & Kovacs G. T. A general purpose system for long-term recording from a microelectrode array coupled to excitable cells. J. Neurosci. Methods 48, 115-121 (1993)
14. Vassanelli, S., & Fromherz, P., Neurons from Rat Brain coupled to Transistors. Applied Physics A 65, 85-88 (1997)
15. Zeck, G., & Fromherz, P. Noninvasive neuroelectronic interfacing with synaptically connected snail neurons immobilized on a semiconductor chip. Proceedings of the National Academy of Sciences 98, 10457-10462 (2001).
16. Bonifazi, P., & Fromherz, P. Silicon Chip for Electronic Communication between Nerve Cells by Noninvasive Interfacing and Analog-Digital Processing. Advanced Materials 14, 1190-1193 (2002)
17. Bliss, T. V. P. & Collingridge, G. L. A synaptic model of memory: long-term potentiation in the hippocampus. Nature, 361 31-39 (1993).
18. Paulsen O, Sejnowski T J. Natural patterns of activity and long-term synaptic plasticity. Curr Opin Neurobiol., 10, 172-9 (2000 )
19. Linden, D. J. & Conner, J. A. Long-term synaptic depression. Ann. Rev. Neurosci. 18, 319-357 (1995).
20. Lee, S. H., Lumelsky, N., Studer, L., Auerbach, J. M., & McKay, R. D. Efficient generation of midbrain and hindbrain neurons from mouse embryonic stem cells. Nat Biotechnol. 18, 675-9 (2000).
21. Kawasaki, H., Suemori, H., Mizuseki, K., Watanabe, K., Urano, F., Ichinose, H., Haruta, M., Takahashi, M., Yoshikawa, K., Nishikawa, S., Nakatsuji, N. & Sasai, Y. Generation of dopaminergic neurons and pigmented epithelia from primate ES cells by stromal cell-derived inducing activity. Proc Natl Acad Sci U S A.;99, 1580-5 (2002).
22. Westmoreland, J. J. Hancock, C. R. and Condie, B. G. Neuronal development of embryonic stem cells: a model of GABAergic neuron differentiation. Biochem Biophys Res Commun. 284, 674-80 (2001).
23. Chang, J. C., Brewer, G. J. & Wheeler, B. C. Modulation of neural network activity by patterning. Biosensors & Bioelectronics 16 527-533 (2001)
24. Nakamura, F., Kalb, R. G., & Strittmatter, S. M. Molecular basis of semaphorin-mediated axon guidance. J. Neorubiol. 44, 219-229 (2000).
25. Raper, J. A. Semaphorins and their receptors in vertebrates and invertebrates. Curr. Opin. Neurobiol. 10, 88-94 (2000).
26. Tessier-Lavigne, M. & Goodman, C. S. The molecular biology of axon guidance. Science 274, 1123-1133 (1996)
27. Hubel D H, Wiesel T N. Early exploration of the visual cortex. Neuron, 20, 401-412 (1998)
28. Cutland, N. Computability, Cambridge University Press, (1980).

1. Device for image processing and learning comprising at least a “multi electrode array” (MEA), over which an homogeneous culture of interconnected neurons, so that forming a cell network, is grown on, wherein said MEA is able to stimulate and record the electric activity of said neurons.

2. Method for parallel processing a digital image comprising the following steps:
a) mapping a digital image (I_{1,2}(x,y)) (INPUT) having a resolution of 1 or 2 bit (I₁(x,y)) or I₂(x,y)) in the case the image is of 1 or 2 bit respectively) of N×N pixel in voltage pulses of 2 or 4 intensity levels applied to a matrix of N×N integrated electrodes on a multi-electrode array (MEA), where spontaneaously interconnected neurons, so that forming a cell network, are maintained in culture;
b) elaborating the image from said neurons by means of the kernel of convolution:
h(ρ,σ,t)=A(t)exp((ρ−ρ(t))/2σ(t)²) (1)
ρ²=x²+y²
c) registering the electric activity of said neurons by means of extracellular MEA electric signals (by voltage) and
d) revealing, for each single electrode and in subsequent time intervals, spikes or firings associated to action potentials generated by said neurons.

3. Method according to claim 2 wherein the firing rate FR(x,y,t) (OUTPUT), measured by the electrode in position (x,y) and during a time interval centered in t, is recorded.

4. Method according to claim 3 wherein the INPUT and the OUTPUT are related by the equation:
FR(x,y,t)=I_{1,2}(x,y)**h(ρ,σ,t) (2)
where ** indicates a two-dimensional convolution.

5. Method according to claim 2 wherein the INPUT digital image (I₈(x,y)) is defined by 8 bit and is divided into 4 or 8 images (Iₘᵢ), each having 2 or 1 bit respectively, where m is 2 or 1 respectively, according to the equation:
$${I}_{8} ( x , y ) = {{{\sum }_{i}}_{1}}^{8 / m} {I}_{mi} {2}^{m ( i - 1 )} \qquad ( 3 )$$
and each single image Iₘᵢ is filtered indipendently and then reassembled in an unique 8 bit image, wherein the whole process of dividing, filtering and reassembling is according to the equation:
$${{{\sum }_{i}}_{1}}^{8 / m} {2}^{m ( i - 1 )} {I}_{mi} ** h ( ρ , σ , t ) \qquad ( 4 )$$
so that the 8 bit image I₈(x,y) is processed with a 8 bit resolution.

6. Method for digital image processing and learning comprising the following steps:
a) stimulate a matrix of N×N electrodes on a multi-electrode array (MEA), where spontaneaosly interconnected neuronal cells, so that forming a cell network, are maintained in culture, by means of a tetanic stimulation composed by bipolar voltage pulses having a frequency of at least 100 Hz, and having at least a pair of non colinear segments (I_{1,2}(x,y)) (INPUT), in order to induce learning or potentiation;
b) measuring the firing rate FR_{1,2}(x,y,t) evoked by the INPUT image;
c) processing the INPUT image as a 8 bit image according to the equation:
$${\sum }_{{1}^{i}}^{8 / m} {2}^{m ( i - 1 )} {FR}_{m , i} ( x , y ) \qquad ( 5 )$$
where FR_{m,i} (x,y) is the measured response to Iₘᵢ(x,y) after the tetanization.

7. Method for digital image processing and learning according to claim 6 wherein the INPUT image is larger than 1000×1000 pixel.
